(19) United States
(12) Patent Application Publication
(10) Pub. No.: US 2008/0116789 A1
(43) Pub. Date: May 22, 2008

(54) 1, 5-NAPHTHYRIDINE COMPOUND AND ORGANIC LIGHT-EMITTING DEVICE

(75) Inventors: Tomona Yamaguchi, Tokyo (JP); Hiroki Ohrui, Kawasaki-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/931,138

(22) Filed: Oct. 31, 2007

(30) Foreign Application Priority Data
Nov. 20, 2006 (JP) 2006-312826

(51) Int. Cl. H01L 27/28; C07D 471/04

(52) U.S. Cl. 313/504; 546/122

(57) ABSTRACT

The present invention provides a novel 1,5-naphthyridine compound represented by the following general formula [I]:

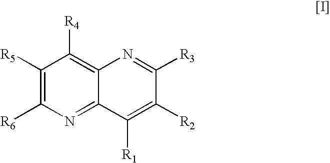

wherein R₁, R₂, R₄ and R₅ each represent one selected from a hydrogen atom, a substituted or unsubstituted alkyl group, and the like; and R₃ and R₆ each represent one selected from a substituted or unsubstituted aralkyl group, a substituted or unsubstituted aryl group, and the like.

### BACKGROUND OF THE INVENTION

1. Field of the Invention

The present invention relates to a novel organic compound and an organic light-emitting device using the same.

2. Description of the Related Art

The recent progress of an organic light-emitting device is significant, and the device suggests its potential to find use in a wide variety of applications because of the following reasons. The device shows a high luminance at a low applied voltage. In addition, the device has features of a variety of emission wavelengths and high-speed responsiveness. Further, the device can be a thin, light-weight light-emitting device.

However, at present, improvements in initial characteristics such as a luminous efficiency, and duration characteristics such as resistance to luminance degradation due to long-term light emission have been needed. Those initial characteristics and duration characteristics result from all layers constituting the device including a hole injection layer, a hole transport layer, a light-emitting layer, a hole blocking layer, an electron transport layer, and an electron injection layer and the like.

Examples of conventionally known materials to be used in the hole blocking layer, the electron transport layer, and the electron injection layer include a phenanthroline compound, an aluminum quinolinol complex, an oxadiazole compound, and a triazole compound. Examples of use of those materials in a light-emitting layer or an electron transport layer are disclosed by Japanese Patent Application Laid-Open Nos. H05-331459, H07-082551, 2001-267080, 2001-131174, H02-216791 and H10-233284, and U.S. Pat. Nos. 4,539,507 and 4,720,432 and 4,885,211. However, the initial characteristics and duration characteristics of an electroluminescence (EL) device of each of those documents are not sufficient.

### SUMMARY OF THE INVENTION

An object of the present invention is to provide a novel 1,5-naphthyridine compound.

Another object of the present invention is to provide an organic light-emitting device having a high emission luminance and a high luminous efficiency by using the novel 1,5-naphthyridine compound. Another object of the present invention is to provide an organic light-emitting device having high durability and small luminance degradation in long-term light emission.

In other words, the present invention provides a 1,5-naphthyridine compound represented by any one of the following general formulae [I] to [III]:

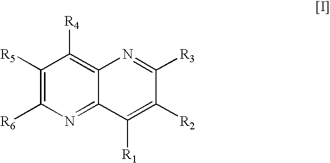

wherein R₁, R₂, R₃, R₄, R₅ and R₆ each represent a group selected from the group consisting of a hydrogen atom, a substituted or unsubstituted alkyl group, a substituted or unsubstituted aralkyl group, a substituted or unsubstituted aryl group, a substituted or unsubstituted heterocyclic group, a substituted or unsubstituted condensed polycyclic aromatic group, a substituted or unsubstituted condensed polycyclic heterocyclic group, a substituted or unsubstituted aryloxy group, a substituted amino group, a halogen atom, a trifluoromethyl group, and a cyano group; and R₁, R₂, R₃, R₄, R₅ and R₆ may be the same as or different from one another, provided that at least two of R₁, R₂, R₃, R₄, R₅ and R₆ each represent a group selected from the group consisting of a substituted or unsubstituted aralkyl group, a substituted or unsubstituted aryl group, a substituted or unsubstituted heterocyclic group, a substituted or unsubstituted condensed polycyclic aromatic group, a substituted or unsubstituted condensed polycyclic heterocyclic group, a substituted or unsubstituted aryloxy group, and a substituted amino group.

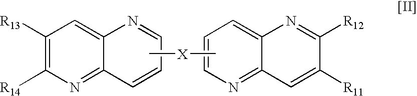

wherein R₁₁, R₁₂, R₁₃ and R₁₄ each represent a group selected from the group consisting of a hydrogen atom, a substituted or unsubstituted alkyl group, a substituted or unsubstituted aralkyl group, a substituted or unsubstituted aryl group, a substituted or unsubstituted heterocyclic group, a substituted or unsubstituted condensed polycyclic aromatic group, a substituted or unsubstituted condensed polycyclic heterocyclic group, a substituted or unsubstituted aryloxy group, a substituted amino group, a halogen atom, a trifluoromethyl group, and a cyano group: and R₁₁, R₁₂, R₁₃ and R₁₄ may be the same as or different from one another; and X represents a group selected from the group consisting of a divalent, substituted or unsubstituted arylene group, a divalent, substituted or unsubstituted heterocyclic group, a divalent, substituted or unsubstituted condensed polycyclic aromatic group, and a divalent, substituted or unsubstituted condensed polycyclic heterocyclic group.

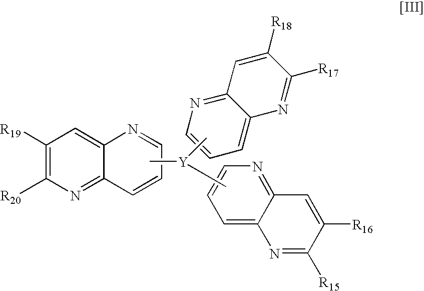

wherein R₁₅, R₁₆, R₁₇, R₁₈, R₁₉ and R₂₀ each represent a group selected from the group consisting of a hydrogen atom, a substituted or unsubstituted alkyl group, a substituted or unsubstituted aralkyl group, a substituted or unsubstituted aryl group, a substituted or unsubstituted heterocyclic group, a substituted or unsubstituted condensed polycyclic aromatic group, a substituted or unsubstituted condensed polycyclic heterocyclic group, a substituted or unsubstituted aryloxy group, a substituted amino group, a halogen atom, a trifluoromethyl group, and a cyano group; and R₁₅, R₁₆, R₁₇, R₁₈, R₁₉ and R₂₀ may be the same as or different from one another; and Y represents a trivalent, substituted or unsubstituted arylene group.

The organic light-emitting device using the 1,5-naphthyridine compound of the present invention provides light emission having a high luminance at a low applied voltage, and is excellent in durability. An organic layer containing the 1,5-naphthyridine compound of the present invention is excellent particularly as an electron transport layer and an excellent light-emitting layer.

Further, the device can be produced by vacuum deposition method, a casting method, or the like. A light-emitting device having a large area can be easily produced at a relatively low cost.

Further features of the present invention will become apparent from the following description of exemplary embodiments with reference to the attached drawings.

### BRIEF DESCRIPTION OF THE DRAWINGS

FIG. 1 is a sectional view illustrating an example of an organic light-emitting device of the present invention.

FIG. 2 is a sectional view illustrating another example of the organic light-emitting device of the present invention.

FIG. 3 is a sectional view illustrating another example of the organic light-emitting device of the present invention.

FIG. 4 is a sectional view illustrating another example of the organic light-emitting device of the present invention.

FIG. 5 is a sectional view illustrating another example of the organic light-emitting device of the present invention.

FIG. 6 is a sectional view illustrating another example of the organic light-emitting device of the present invention.

FIG. 7 is a sectional view illustrating another example of the organic light-emitting device of the present invention.

FIG. 8 is a sectional view illustrating another example of the organic light-emitting device of the present invention.

### DESCRIPTION OF THE EMBODIMENTS

Hereinafter, the present invention will be described in detail.

First, a 1,5-naphthyridine compound of the present invention will be described.

The 1,5-naphthyridine compound of the present invention is represented by any one of the above general formulae [I] to [III]. In addition, out of the compounds each represented by the general formula [I], a compound in which R₁, R₂, R₄ and R₅ each represent a group selected from a hydrogen atom, a substituted or unsubstituted alkyl group, a halogen atom, a trifluoromethyl group, and a cyano group, and R₃ and R₆ each represent a group selected from a substituted or unsubstituted aralkyl group, a substituted or unsubstituted aryl group, a substituted or unsubstituted heterocyclic group, a substituted or unsubstituted condensed polycyclic aromatic group, a substituted or unsubstituted condensed polycyclic heterocyclic group, a substituted or unsubstituted aryloxy group, and a substituted amino group is preferable.

Specific examples of the substituents in the general formulae [I] to [III] will be shown below.

As the alkyl group, a methyl group, an ethyl group, an n-propyl group, an iso-propyl group, an n-butyl group, a ter-butyl group, an octyl group, and the like can be given.

As the aralkyl group, a benzyl group, a phenethyl group, and the like can be given.

As the aryl group, a phenyl group, a biphenyl group, a terphenyl group, and the like can be given.

As the heterocyclic group, a thienyl group, a pyrrolyl group, a pyridyl group, a bipyridyl group, a terpyridyl group, an oxazolyl group, an oxadiazolyl group, a thiazolyl group, a thiadiazolyl group, and the like can be given.

As the condensed polycylic aromatic group, a fluorenyl group, a naphthyl group, a fluoranthenyl group, an anthryl group, a phenanthryl group, a pyrenyl group, a tetracenyl group, a pentacenyl group, a perylenyl group, a triphenylenyl group, and the like can be given.

As the condensed polycyclic heterocyclic group, a quinolyl group, a quinoxalyl group, a carbazolyl group, an acridinyl group, a phenazyl group, a phenanthrolyl group, a benzoxazolyl group, a benzthiazolyl group, and the like can be given.

As the aryloxy group, a phenoxyl group, a fluorenoxyl group, a naphthoxyl group, and the like can be given.

As the substituted amino group, a dimethylamino group, a diethylamino group, a dibenzylamino group, a diphenylamino group, a ditolylamino group, a dianisolylamino group, a fluorenylphenylamino group, a difluorenylamino group, a naphthylphenylamino group, a dinaphthylamino group, and the like can be given.

As the halogen atom, fluorine, chlorine, bromine, iodine, and the like can be given.

As the divalent arylene group, a phenylene group, a biphenylene group, a terphenylene group, and the like can be given.

As the divalent heterocyclic group, a furylene group, a pyrolylene group, a pyridylene group, a terpyridylene group, a thienylene group, a terthienylene group, an oxazolylene group, a thiazolylene group, and the like can be given.

As the divalent condensed polycylic aromatic group, a naphthylene group, a fluorenylene group, an anthracenylene group, a pyrenylene group, a triphenylene group, and the like can be given.

As the divalent condensed polycyclic heterocyclic group, a quinolylene group, a quinoxalylene group, a carbazolylene group, a phenanthrylene group, a thiophenylene group, a pyridylene group, a pyrazilene group, a pyrimidylene group, a pyridazylene group, a benzoxazoylene group, a benzothiazolylene group, a phenadylene, and the like can be given.

As the trivalent arylene, a phenylene group, a triphenylene group, and the like can be given.

The above-mentioned substituents may further have following substituents: alkyl groups such as a methyl group, an ethyl group, an n-propyl group, an iso-propyl group, an n-butyl group, a ter-butyl group, and an octyl group; aralkyl groups such as a benzyl group, and a phenethyl group; aryl groups such as a phenyl group, a biphenyl group, and a terphenyl group; heterocyclic groups such as a thienyl group, a pyrrolyl group, a pyridyl group, a bipyridyl group, a terpyridyl group, an oxazolyl group, an oxadiazolyl group, a thiazolyl group, and a thiadiazolyl group; condensed polycyclic aromatic groups such as a fluorenyl group, a naphthyl group, a fluoranthenyl group, an anthryl group, a phenanthryl group, a pyrenyl group, a tetracenyl group, a pentacenyl group, a perylenyl group, and a triphenylenyl group; condensed polycyclic heterocyclic groups such as a quinolyl group, a carbazolyl group, an acridinyl group, a phenazyl group, and a phenanthrolyl group; aryloxy groups such as a phenoxyl group, a fluorenoxyl group, and a naphthoxyl group; substituted amino groups such as a dimethylamino group, a diethylamino group, a dibenzylamino group, a diphenylamino group, a ditolylamino group, a dianisolylamino group, a fluorenylphenylamino group, a difluorenyl group, a naphthylphenylamino group, and a dinaphthylamino group; halogen atoms such as fluorine, chlorine, bromine, and iodine; trifluoromethyl groups; cyano groups; and the like.

Next, representative examples of the 1,5-naphthyridine compound of the present invention will be given below. However, the present invention is not limited to those examples.

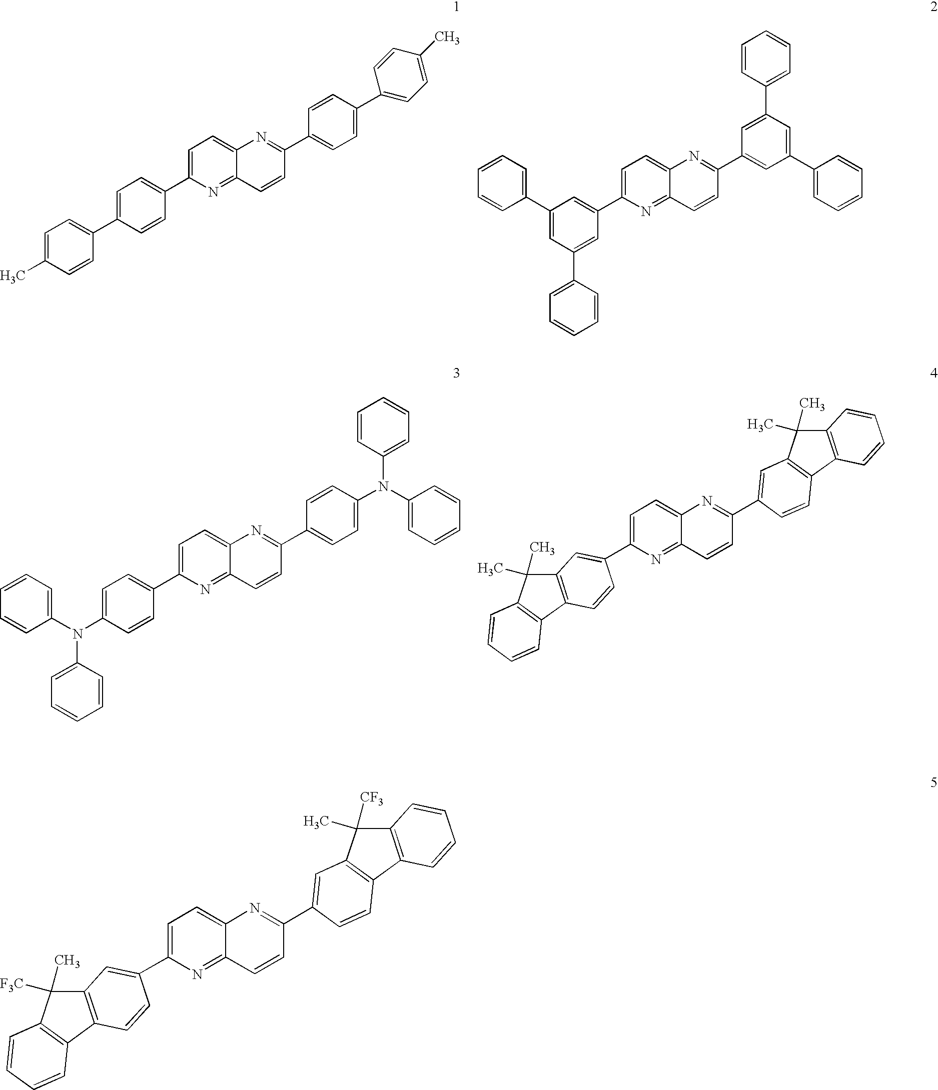

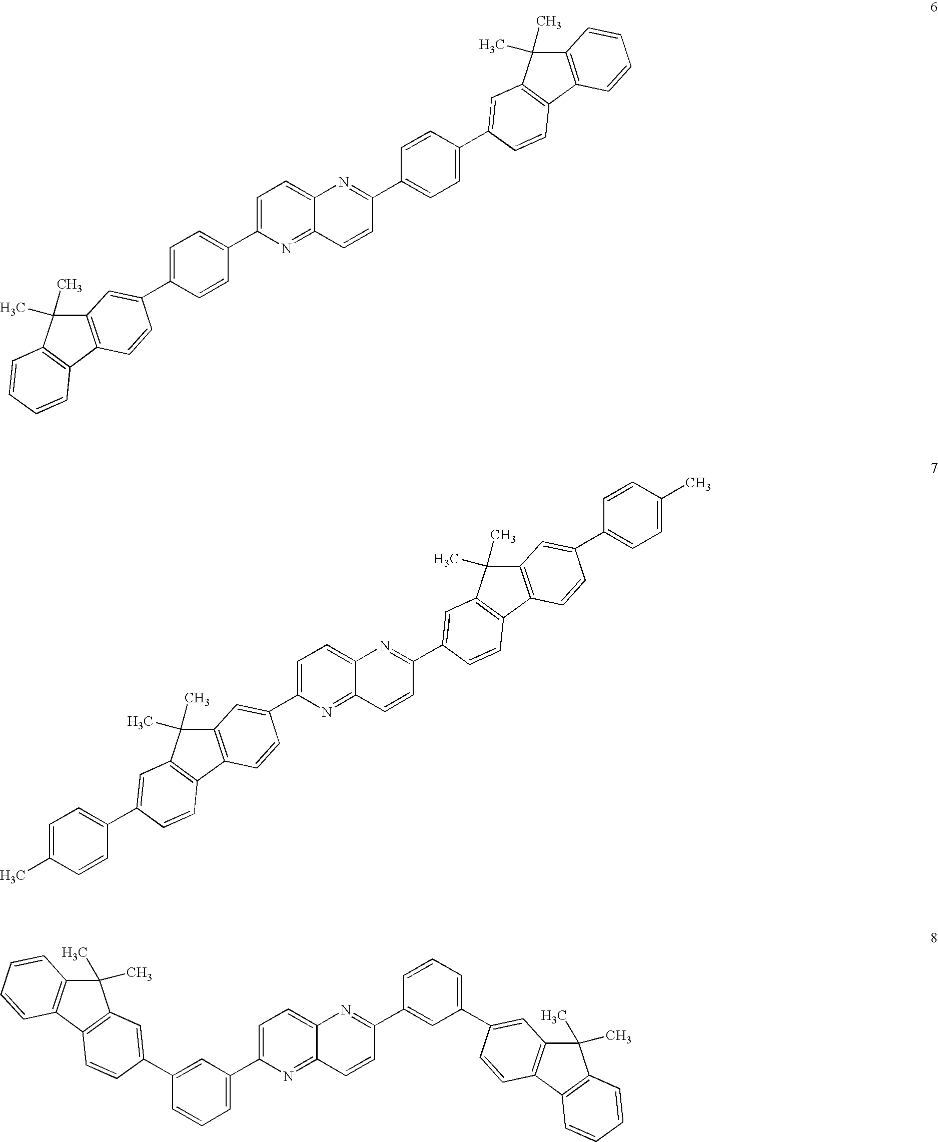

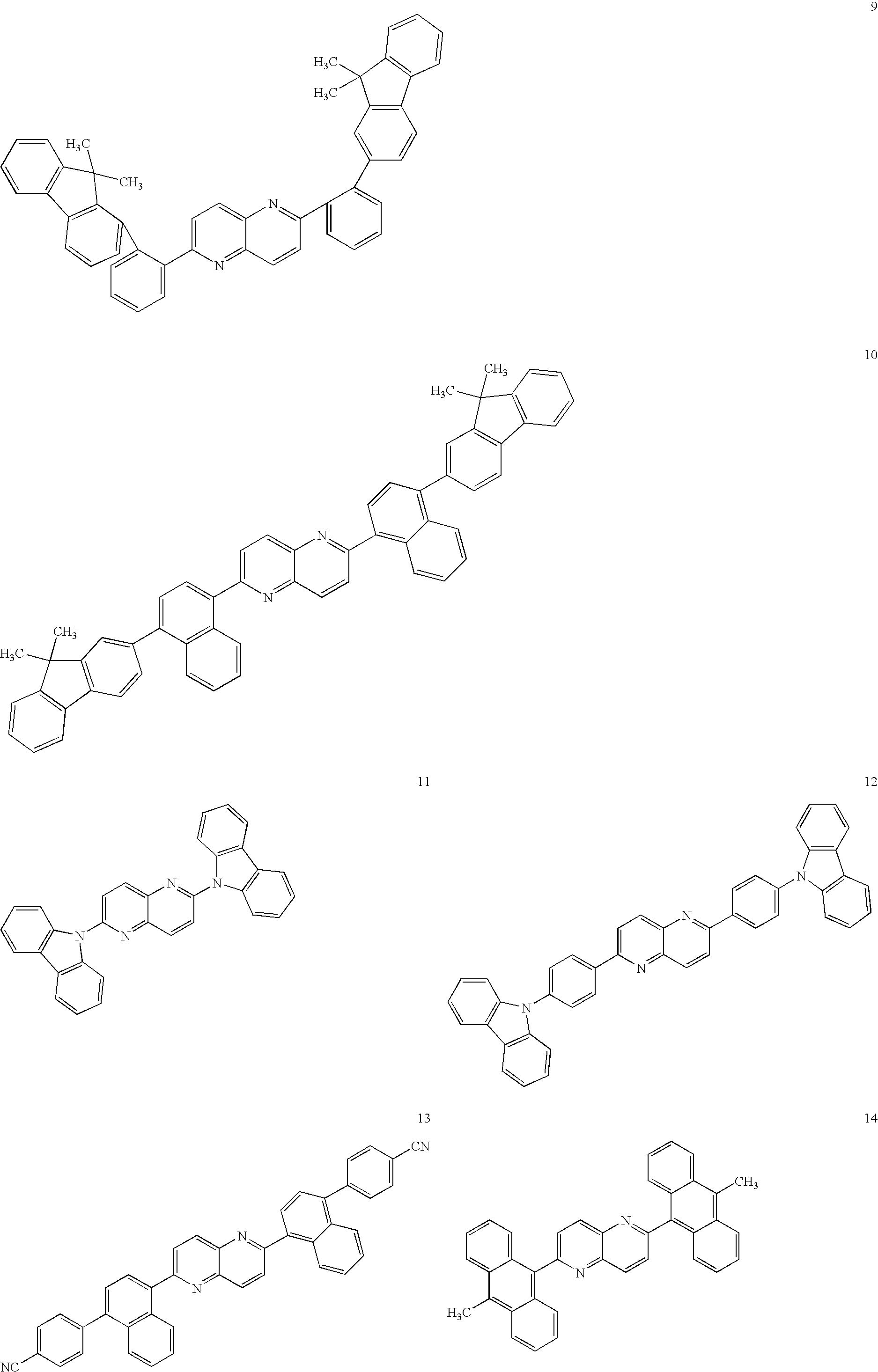

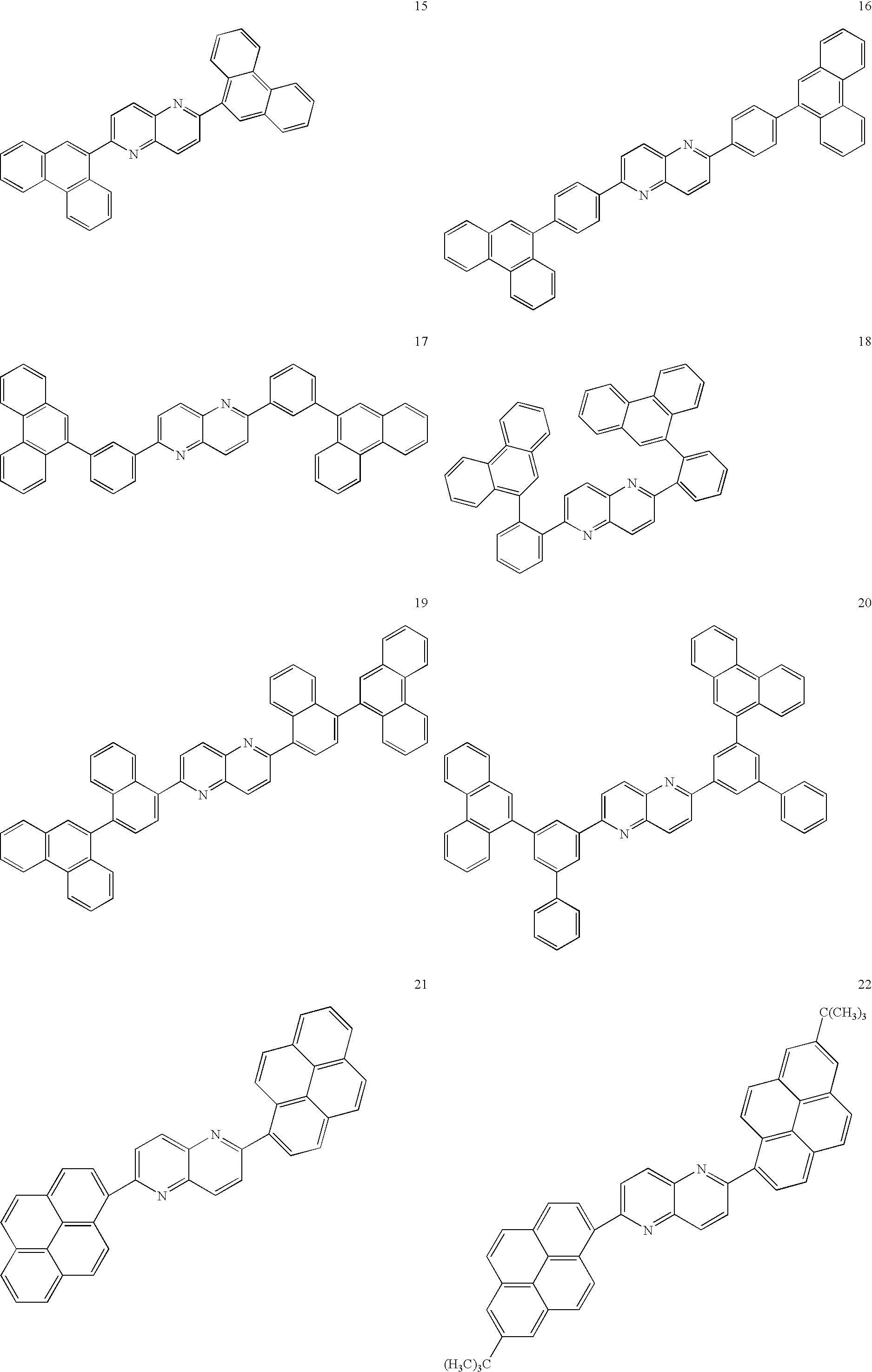

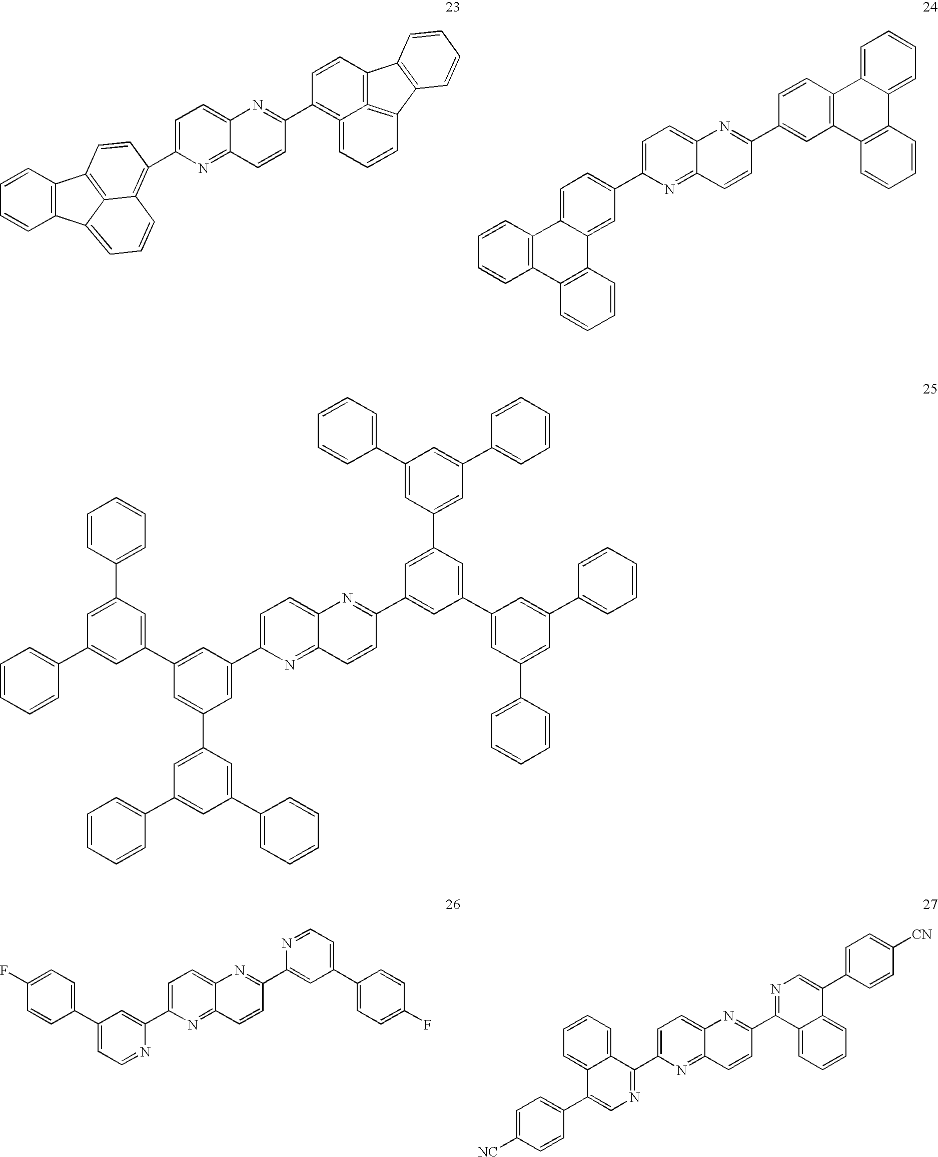

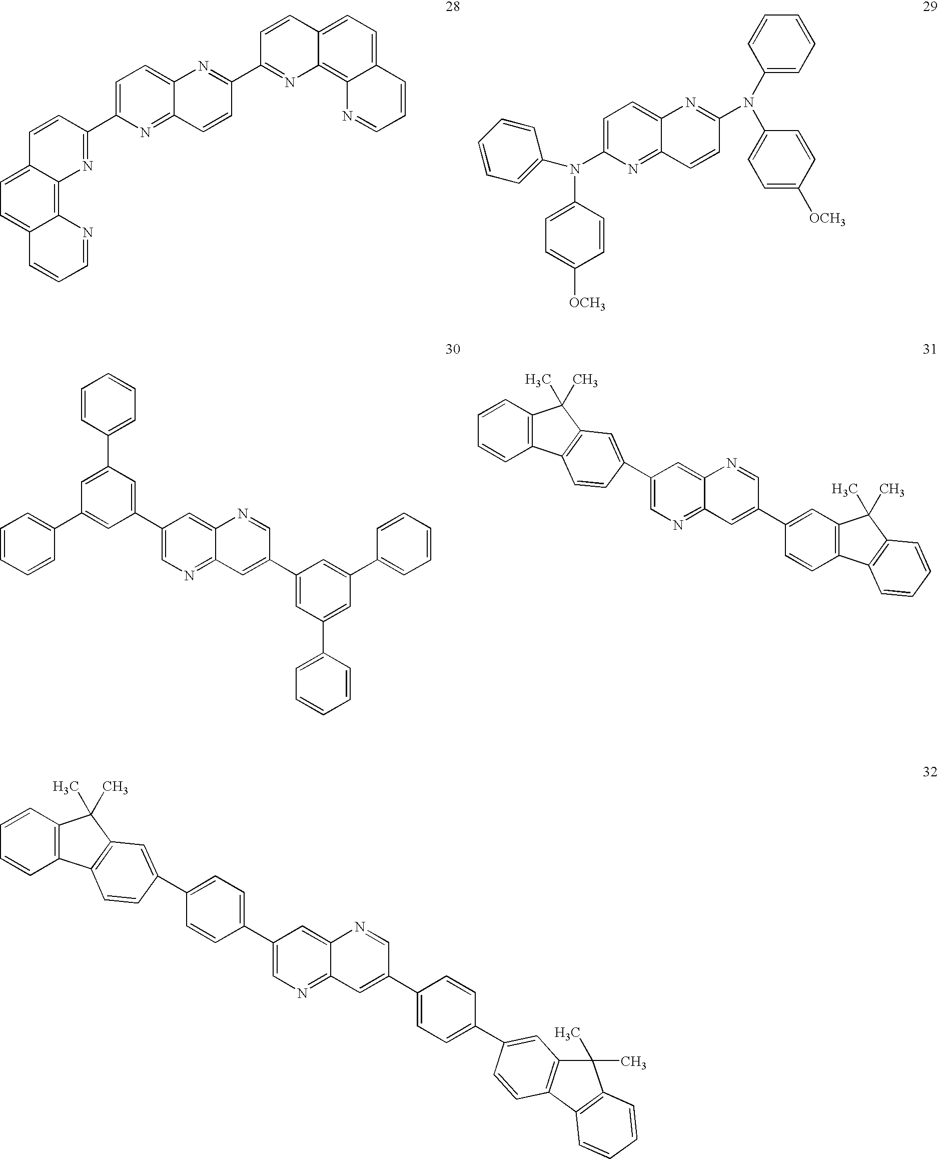

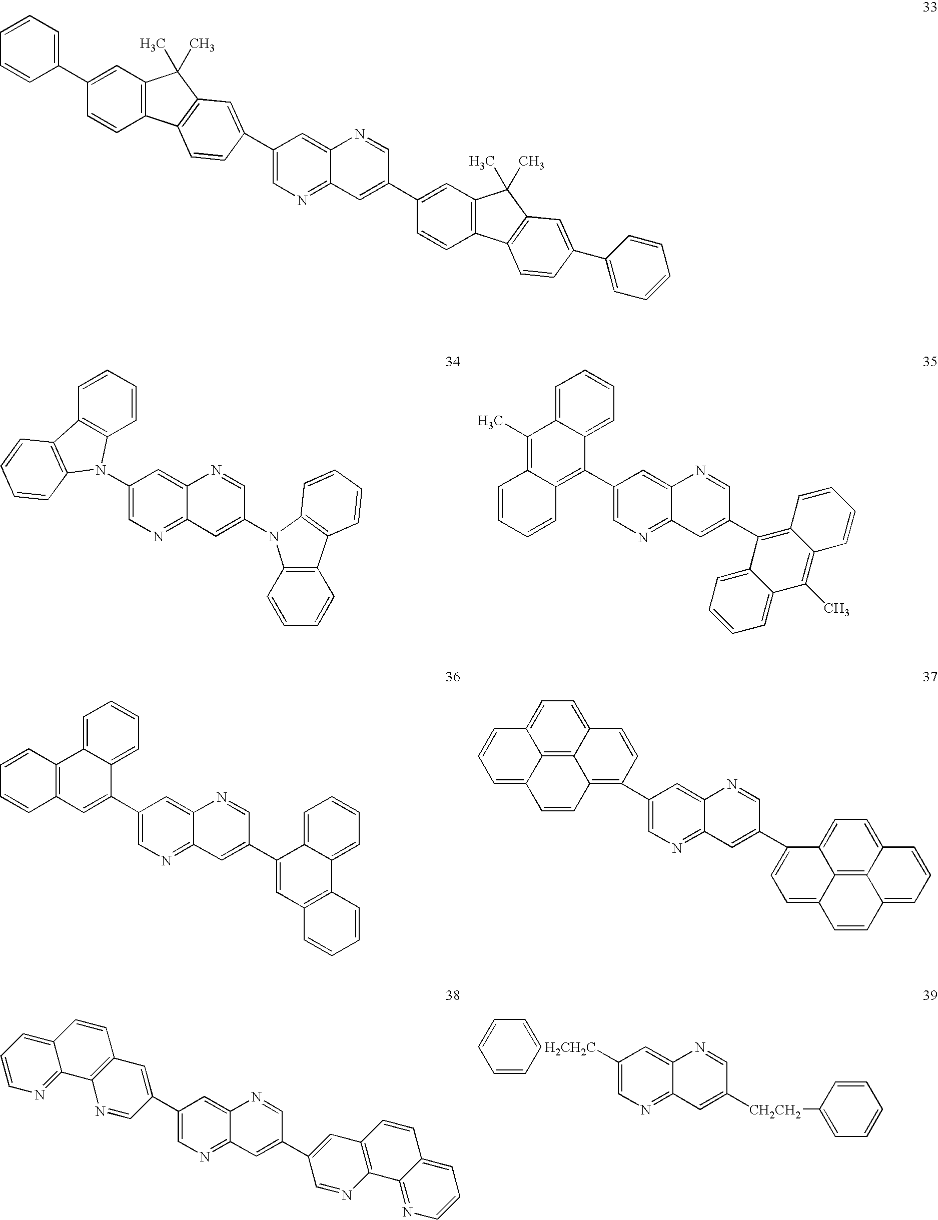

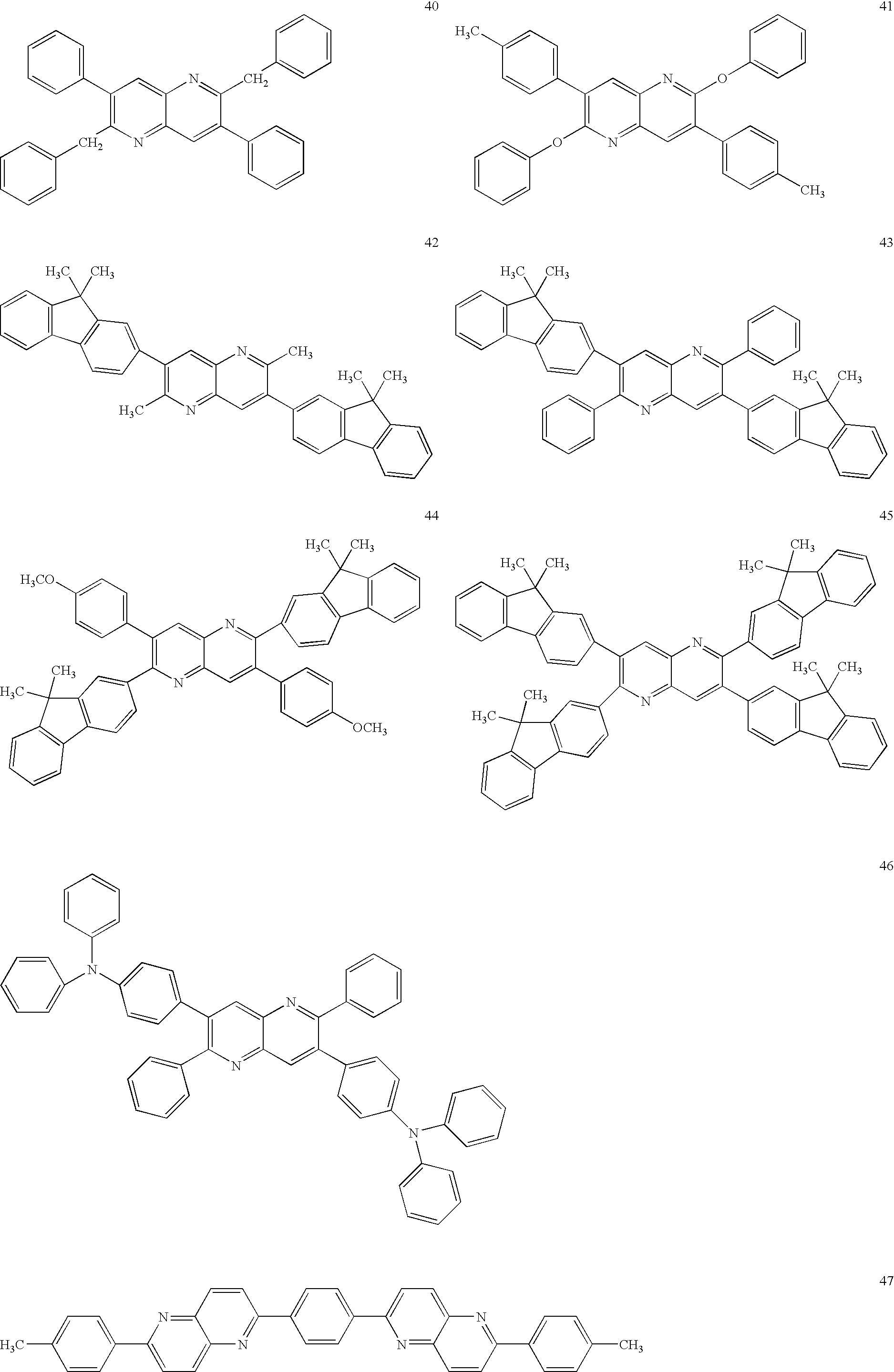

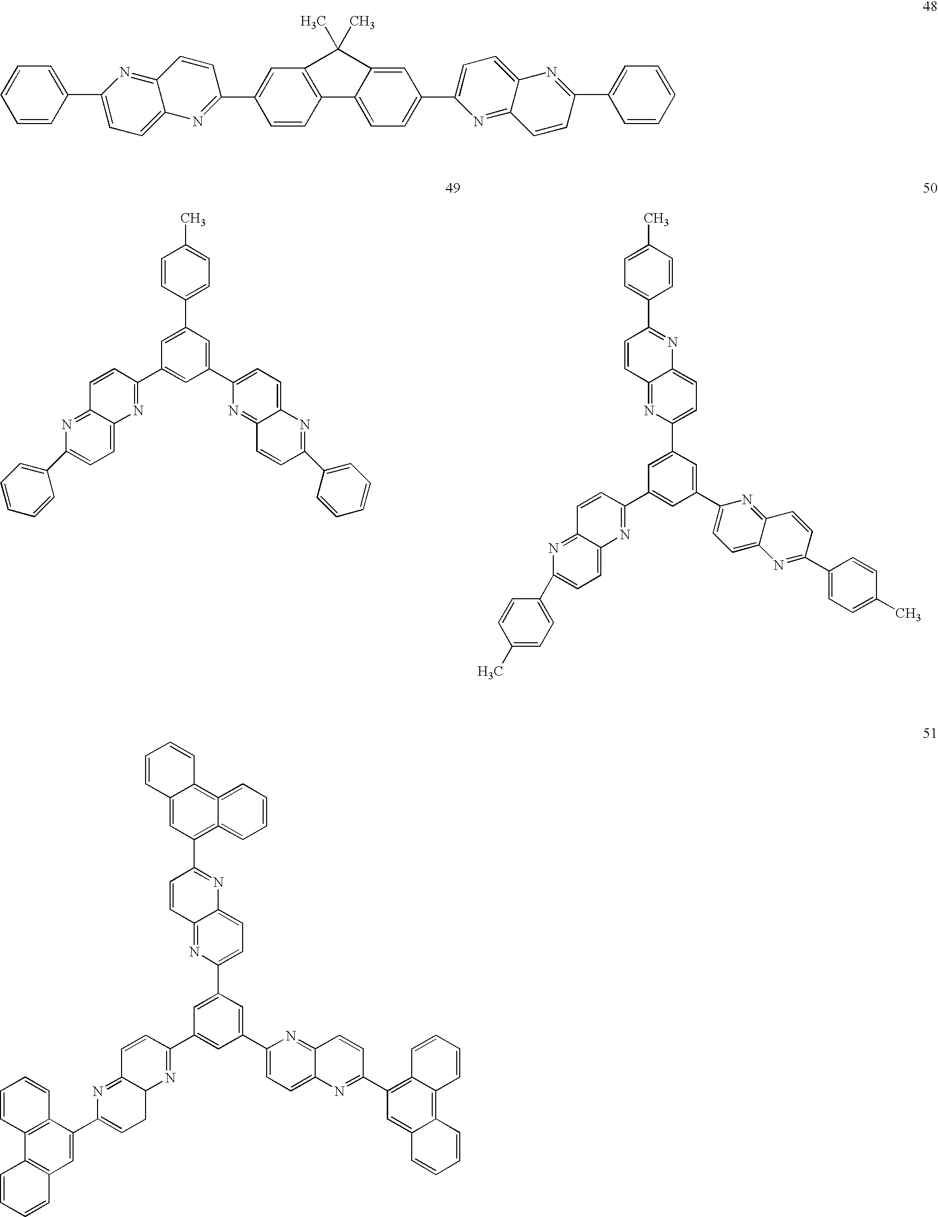

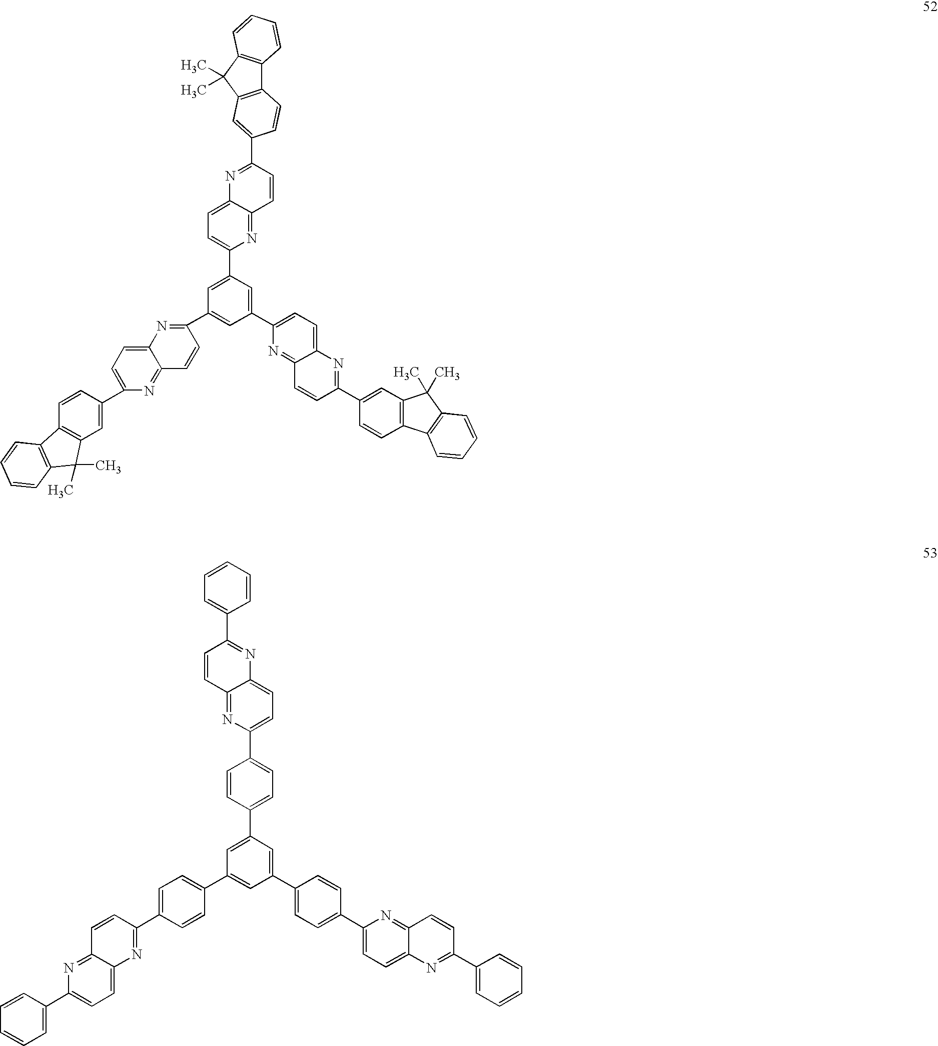

The 1,5-naphthyridine compound of the present invention can be synthesized by a generally known method. A 1,5-naphthyridine compound intermediate is obtained by any one of the methods described in, for

example, the following documents. Then, the target product can be obtained by employing a synthesis method such as a Suzuki Coupling method (Chem. Rev., 95, 2457 (1995)) involving the use of a palladium catalyst.
J. Org. Chem., 33, 1384 (1968)
J. Chem. Soc., 1879 (1954)
J. Org. Chem., 46, 833 (1981)

The 1,5-naphthyridine compound of the present invention is superior to a conventional compound in electron-transporting property, light-emitting property, and durability, and is useful as a layer containing an organic compound in an organic light-emitting device, in particular, as an electron transport layer and a light-emitting layer. In addition, a layer formed of the compound by means of a vacuum deposition method, a solution application method, or the like hardly causes crystallization or the like, and is excellent in stability with elapse of time.

Next, the organic light-emitting device of the present invention will be described in detail.

The organic light-emitting device of the present invention includes at least a pair of electrodes formed of an anode and a cathode, and one or more layers each containing an organic compound interposed between the pair of electrodes. In this light-emitting device, at least one layer of the layers each containing the organic compound contains at least one kind of the 1,5-naphthyridine compound of the present invention.

At least an electron transport layer or a light-emitting layer out of the one or more layers each containing an organic compound of the organic light-emitting device of the present invention preferably contains at least one kind of the 1,5-naphthyridine compound. A compound having a relatively low HOMO out of the 1,5-naphthyridine compounds has high hole-blocking property, and is particularly preferably used in an electron transport layer and also preferably used in a hole-blocking layer.

The layer containing the 1,5-naphthyridine compound of the present invention can be formed between the anode and the cathode by means of a vacuum deposition method or a solution application method. The thickness of the organic layer is thinner than 10 μm, and the layer is formed into a thin film having a thickness of preferably 0.5 μm or less, or more preferably 0.01 to 0.5 μm.

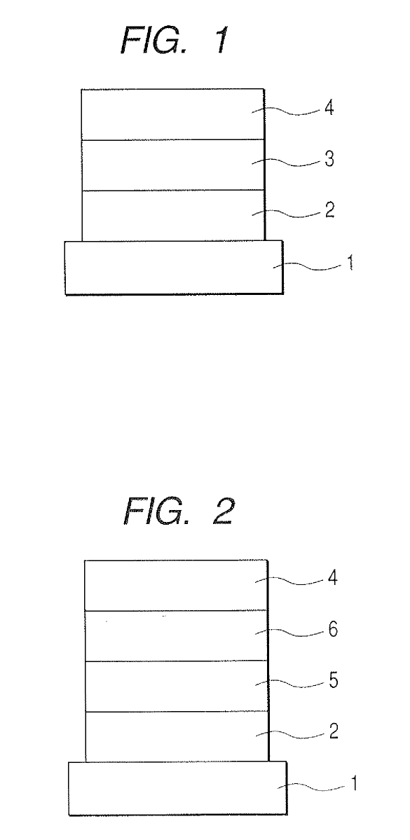

FIGS. 1 to 8 illustrate preferable examples of the organic light-emitting device of the present invention.

FIG. 1 is a sectional view illustrating an example of the organic light-emitting device of the present invention. FIG. 1 illustrates a constitution in which the anode 2, the light-emitting layer 3, and the cathode 4 are sequentially provided onto the substrate 1. The light-emitting device to be used here is useful for the case where one compound used in the device itself has hole-transporting ability, an electron-transporting ability and light-emitting property, or the case where compounds having the respective properties are used as a mixture.

FIG. 2 is a sectional view illustrating another example of the organic light-emitting device of the present invention. FIG. 2 illustrates a constitution in which the anode 2, the hole transport layer 5, the electron transport layer 6, and the cathode 4 are sequentially provided onto the substrate 1. In this case, a material having one or both of hole-transporting property and electron-transporting property is used as a light-emitting substance in each layer. This case is useful when the device is used in combination with a mere hole-transporting substance or electron-transporting substance having no light-emitting property. In addition, in this case, a light-emitting layer is composed of one of the hole transport layer 5 and the electron transport layer 6.

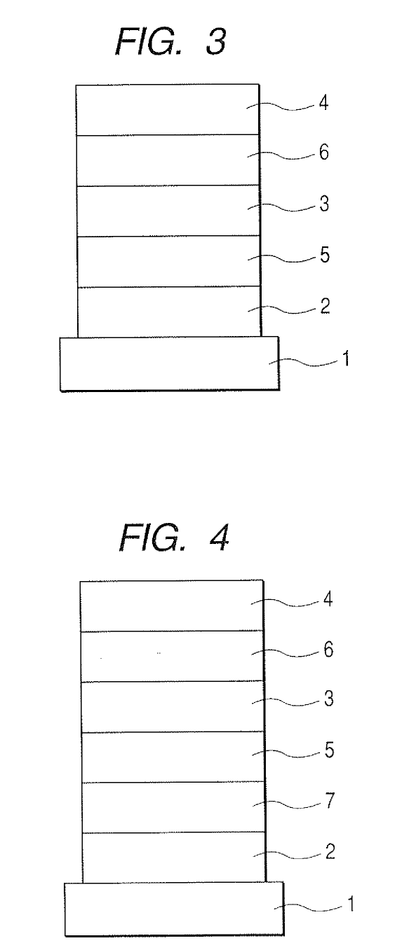

FIG. 3 is a sectional view illustrating another example of the organic light-emitting device of the present invention. FIG. 3 illustrates a constitution in which the anode 2, the hole transport layer 5, the light-emitting layer 3, the electron transport layer 6, and the cathode 4 are sequentially provided onto the substrate 1. This constitution separates a carrier-transporting function and a light-emitting function. In addition, the device is formed by suitably by combining a compound having a hole-transporting property, a compound having an electron-transporting property, and a compound having a light-emitting property, so that the degree of freedom in selection of materials extremely increases. In addition, various compounds different from each other in emission wavelength can be used. As a result, the range of emission colors can be widened. Further, a luminous efficiency can be improved by effectively confining each carrier or exciton in the central light-emitting layer 3.

FIG. 4 is a sectional view illustrating another example of the organic light-emitting device of the present invention. FIG. 4 illustrates the same constitution as that of FIG. 3 except that the hole injection layer 7 is inserted on the side of the anode 2. This constitution has an improving effect on adhesiveness between the anode 2 and the hole transport layer 5 or on hole-injecting property, and is effective in lowering the driving voltage of the device.

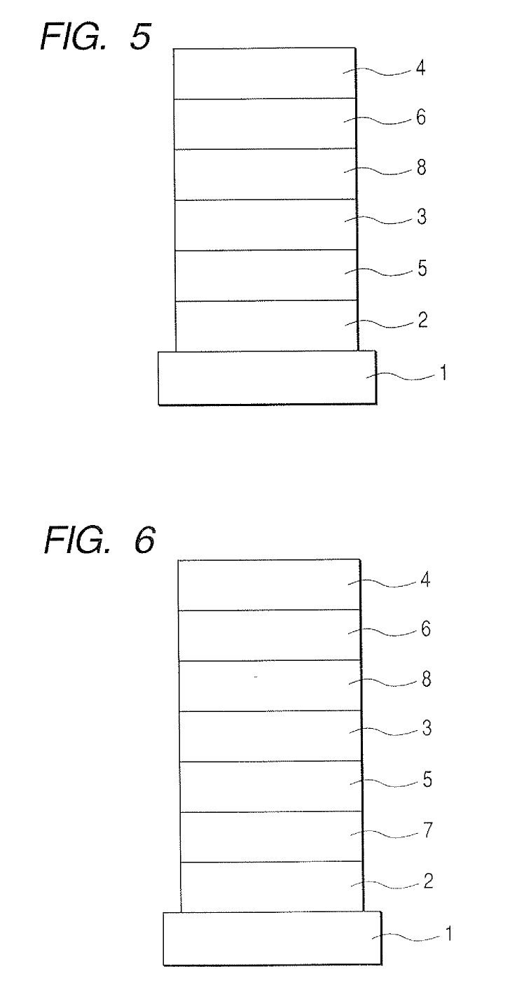

FIGS. 5 and 6 are each a sectional view illustrating another example of the organic light-emitting element of the present invention. FIGS. 5 and 6 illustrate constitutions different from those illustrated in FIGS. 3 and 4, respectively, in that a layer for blocking the escape of a hole or an exciton toward the cathode 4 (hole-blocking layer 8) is inserted between the light-emitting layer 3 and the electron transport layer 6. The constitution illustrated in each of FIGS. 5 and 6 is effective for an improvement in emission efficiency of the organic light-emitting device because a compound having an extremely high ionization potential is used in the hole-blocking layer 8.

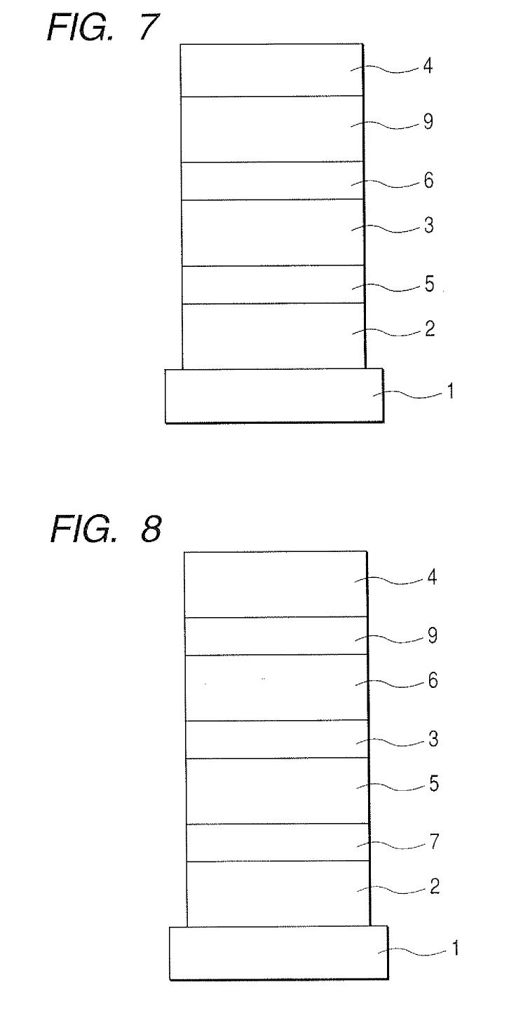

FIGS. 7 and 8 are each a sectional view illustrating another example of the organic light-emitting element of the present invention. FIGS. 7 and 8 show constitutions different from those illustrated in FIGS. 3 and 4, respectively, in that an electron injection layer 9 is inserted between the electron transport layer 6 and the cathode 4. The constitution illustrated in each of FIGS. 7 and 8 has an improving effect on adhesiveness between the cathode 4 and the electron transport layer 6 or on electron injection property, and is effective in lowering the driving voltage of the device.

It should be noted that the device constitutions illustrated in FIGS. 1 to 8 are merely very basic constitutions, and the constitution of an organic light-emitting device using the compound of the present invention is not limited to those constitutions. The device may adopt any one of various layer constitutions. For example, an insulating layer may be provided at an interface between an electrode and an organic layer. Alternatively, an adhesive layer or an interference layer may be provided. Alternatively, a hole transport layer may be composed of two layers different from each other in ionization potential.

The organic light-emitting element of the present invention can use, for example, a conventionally known hole-transporting compound, light-emitting compound, or electron-transporting compound together with the 1,5-naphthyridine compound of the present invention as required.

Examples of those compounds are shown below.

A hole-injecting/transporting material preferably has a property of easily injecting a hole from the anode and an excellent mobility that the injected hole is transported to the light-emitting layer. Low-molecular-weight-based and polymer-based materials each having hole injection/transport property include, but of course not limited to, the following materials: a triarylamine derivative, a phenylenediamine derivative, a triazole derivative, an oxadiazole derivative, an immidazole derivative, a pyrazoline derivative, a pyrazolone derivative, an oxazole derivative, a fluorenone derivative, a hydrazone derivative, a stilbene derivative, a phthalocyanine derivative, a porphyrin derivative, and poly(vinylcarbazole), a poly(silylene), poly(thiophene), and other electrically conductive polymers.

Materials each of which is related to the light-emitting function of the organic light-emitting device include, but of course not limited to, the following compounds: polycyclic condensed aromatic compounds (including naphthalene derivatives, phenanthrene derivatives, fluorene derivatives, pyrene derivatives, tetracene derivatives, coronene derivatives, chrysene derivatives, perylene derivatives, 9,10-diphenylanthracene derivatives, and rubrene); quinacridone derivatives; acridone derivatives; coumarin derivatives; pyran derivatives; Nile red; pyrazine derivatives; benzoimidazole derivatives; benzothiazole derivatives; benzoxazole derivatives; stilbene derivatives; organometallic complexes (including organic aluminum complexes such as tris(8-quinolinolato)aluminum, organic beryllium complexes, organic platinum complexes, and organic iridium complexes); and polymeric derivatives such as poly(phenylenevinylene) derivatives, poly(fluorene) derivatives, poly(phenylene) derivatives, poly(thienylenevinylene) derivatives, and poly(acetylene) derivatives.

An electron-injecting/transporting material to be used in addition to the 1,5-naphthyridine compound of the present invention can be arbitrarily selected from materials having a function of easily injecting an electron from the cathode; and has a function of transporting the injected electron to the light-emitting layer, and the selection is performed while, for example, a balance between the functions of the electron-injecting/transporting material and the carrier mobility of the hole-transporting material is taken into consideration. Materials having electron injection/transport property include, but of course not limited to, the following materials: oxadiazole derivatives, oxazole derivatives, thiazole derivatives, thiadiazole derivatives, pyrazine derivatives, triazole derivatives, triazine derivatives, perylene derivatives, quinoline derivatives, quinoxaline derivatives, fluorenone derivatives, anthrone derivatives, phenanthroline derivatives, and organometallic complexes.

The layer containing the 1,5-naphthyridine compound of the present invention and any other layer containing an organic compound are generally formed into a thin film by vacuum deposition, ionized deposition, sputtering, or plasma. Alternatively, the thin film is formed by a known application method (such as a spin coating method, a dipping method, a cast method, an LB method, or an ink-jet method) involving dissolving a material for the film in a proper solvent. In particular, when the film is formed by the application method, a material for forming a film can be used in combination with a proper binder resin.

The binder resin can be selected from a wide range of binder resins, and examples of the binder resin include, but are not limited to the following: a polyvinyl carbazole resin, a polycarbonate resin, a polyester resin, a polyallylate resin, a polystyrene resin, an acrylic resin, a methacrylic resin, a butyral resin, a polyvinyl acetyl resin, a diallyl phthalate resin, a phenol resin, an epoxy resin, a silicone resin, a polysulfone resin, and a urea resin.

Each of those binder resins may be used alone, or one or more of the binder resins may be mixed as a copolymer.

An anode material having a larger work function is desirable. Examples of an anode material that can be used include: metal elements such as gold, silver, platinum, nickel, palladium, cobalt, selenium, vanadium, and alloys of them; and metal oxides such as tin oxide, zinc oxide, indium tin oxide (ITO), and indium zinc oxide. A conductive polymer such as polyaniline, polypyrrole, polythiophene, or polyphenylene sulfide can also be used. Each of those electrode substances may be used alone, or two or more selected substances may be used in combination.

On the other hand, a cathode material having a smaller work function is desirable. Examples of a cathode material that can be used include metal elements such as lithium, sodium, potassium, cesium, calcium, magnesium, aluminum, indium, silver, lead, tin, and chromium, alloys of two or more of the metal elements, and salts of the metal elements. A metal oxide such as indium tin oxide (ITO) can also be used. In addition, a cathode may have a single layer constitution, or may have a multilayer constitution.

The substrate is not particularly limited; provided that an opaque substrate such as a metal substrate or a ceramic substrate, or a transparent substrate such as glass, quartz, or a plastic sheet is used. In addition, a emission color can be controlled by using a color filter film, a fluorescent color conversion filter film, a dielectric reflective film, or the like as the substrate.

The produced device may be provided with a protective layer or a sealing layer for the purpose of preventing the device from contacting with oxygen, moisture, or the like. Examples of the protective layer include: inorganic material films such as a diamond thin film, a metal oxide, and a metal nitride; polymer films such as a fluorine resin, polyparaxylene, polyethylene, a silicone resin, and a polystyrene resin; and a photocurable resin. In addition, the device itself can be covered with glass, a gas impervious film, metal, or the like, and can be packaged with an appropriate sealing resin.

Hereinafter, the present invention will be described more specifically by way of examples. However, the present invention is not limited to those examples.

### Example 1

### Method of Producing Exemplified Compound No. 4

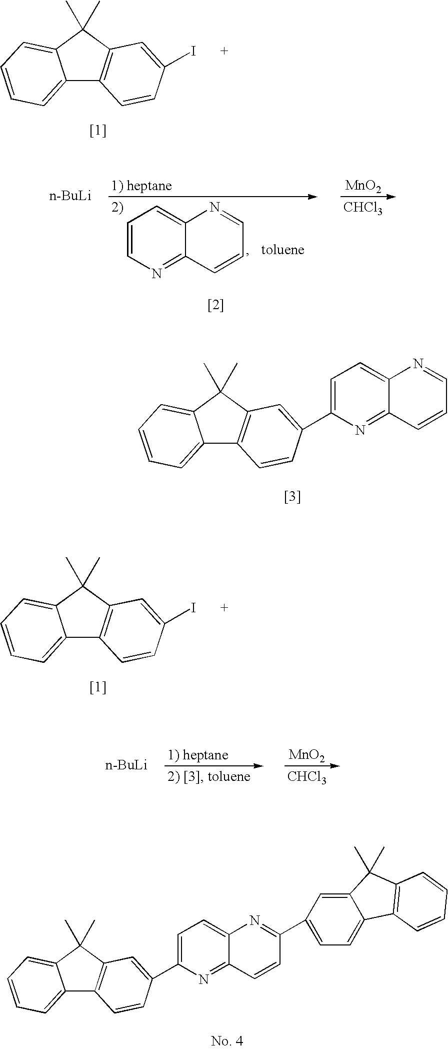

3.7 g (12 mmol) of 2-iodo-9,9-dimethyl-fluorene [1] and 111 ml of heptane were loaded into a 200-ml three-necked flask the inside air of which had been replaced with nitrogen, and the whole was stirred at room temperature, whereby 2-iodo-9,9-dimethyl-fluorene was dissolved in heptane. The dissolved solution was cooled to −40° C., and 7.5 mL (12 mmol) of n-butyllithium/hexane solution (1.58 mol/l) were added to the dissolved solution. The temperature of the mixture was increased to 0° C., and 1.0 g (7.7 mmol) of 1,5-naphthyridine [2] dissolved in 23 mL of toluene was added to the mixture. After that, the temperature of the mixture was gradually increased to room temperature, and then the mixture was stirred for 2.5 hours. Water was added to the reaction solution, and the resultant organic layer was dried with anhydrous sodium sulfate. After that, the solvent was removed by distillation, whereby brown oily substance was obtained. 30 mL of chloroform and 1.4 g (16 mmol) of manganese (IV) oxide were added to the substance, and the whole was stirred at room temperature for 2 hours. The solvent of the filtrate obtained by filtering the reaction solution was removed by distillation, whereby reddish brown oily substance was obtained. The resultant reaction product was separated and purified by means of silica gel column chromatography (mixed developing solvent of chloroform and methanol), whereby 1.6 g of an intermediate compound [3] were obtained (64% yield).

Subsequently, the foregoing reaction was performed in the same manner as that described above except that the compound [2] in the foregoing reaction was replaced with 0.56 g (1.7 mmol) of the compound [3]. The resultant coarse product was separated and purified by means of silica gel column chromatography (mixed developing solvent of chloroform and methanol) and preparative GPC, whereby 290 mg of Exemplified Compound No. 4 (pale yellow crystal) were obtained (32% yield).

### Examples 2 and 3

### Methods of Producing Exemplified Compounds No. 5 and 7

Exemplified Compound No. 5 and 7 are synthesized in the same manner as in Example 1 except that the corresponding iodo bodies are used instead of 2-iodo-9,9-dimethyl-fluorene [1], respectively.

### Example 4

### Method of Producing Exemplified Compound No. 15

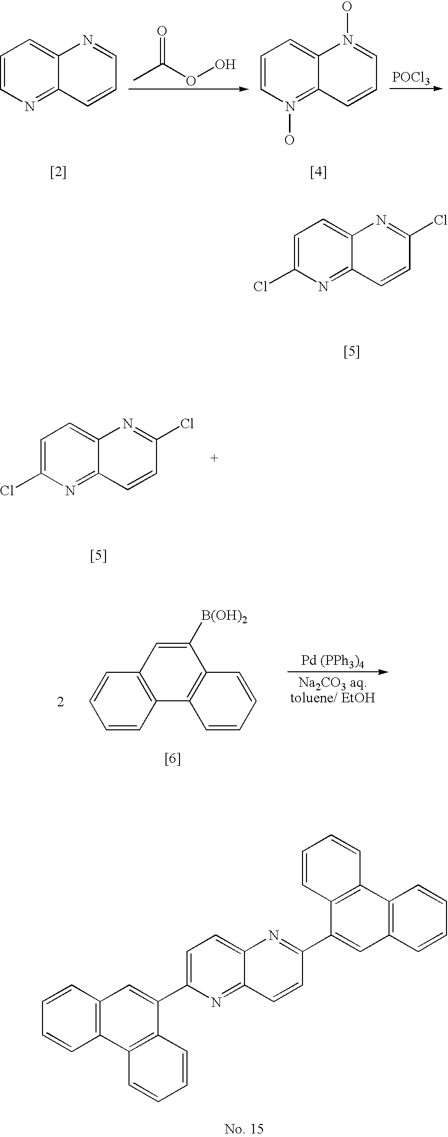

2,6-dichloro-1,5-naphthyridine [5] was obtained in 55% yield by the synthesis method described in J. Chem. Soc., 1879 (1954).

1.1 g (5.6 mmol) of a compound [5], 3.7 g (17 mmol) of phenanthrene-9-boronic acid [6], 200 ml of toluene, and 100 ml of ethanol were loaded into a 500-ml three-necked flask. Then, an aqueous solution of 20 g of sodium carbonate in 100 ml of water was dropped to the mixture while the mixture was stirred in a nitrogen atmosphere at room temperature. Next, 0.33 g (0.29 mmol) of tetrakis(triphenylphosphine)palladium(0) was added to the mixture, and the mixture was stirred at room temperature for 30 minutes. After that, the temperature of the mixture was increased to 77° C., and the mixture was stirred for 5 hours. After the reaction, the organic layer was extracted with chloroform, dried with anhydrous sodium sulfate, and purified with a silica gel column (mixed developing solvent of toluene and ethyl acetate), whereby 1.8 g of Exemplified Compound No. 15 (pale yellow crystal) were obtained (67% yield).

### Examples 5 to 13

### Methods of Producing Exemplified Compounds No. 16 to 24

Exemplified Compounds No. 16 to 24 are each synthesized in the same manner as in Example 4 except that the corresponding boronic acids are used instead of phenanthrene-9-boronic acid [6], respectively.

### Example 14

### Method of Producing Exemplified Compound No. 31

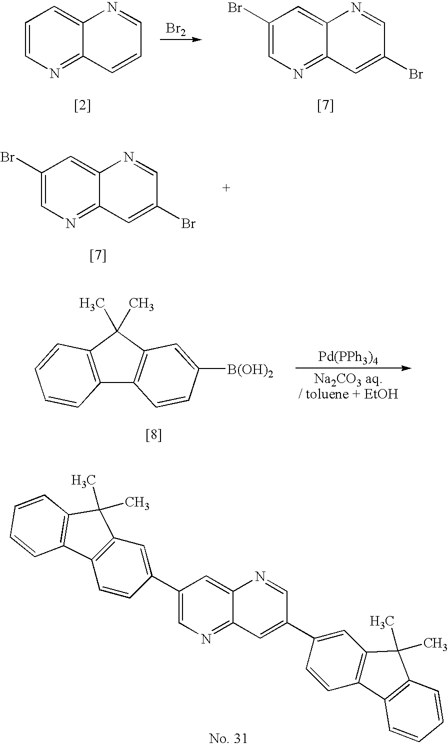

3,7-dibromo-1,8-naphthyridine [7] (white crystal) was obtained in 8% yield by the synthesis method described in J. Org. Chem., 33, 1384 (1968).

280 mg of Exemplified Compound No. 31 (pale yellow crystal) were obtained from the following compounds (45% yield) by a Suzuki Coupling reaction similar to the method described in Example 4. 330 mg (1.2 mmol) of 3,7-dibromo-1,5-naphthyridine [7] 1.1 g (4.0 mmol) of 9,9-dimethylfluorene-2-boronic acid [8]

### Examples 15 to 21

### Methods of Producing Exemplified Compounds No. 30, 33 and 34 to 38

Exemplified Compounds No. 30, 33 and 34 to 38 are each synthesized in the same manner as in Example 14 except that the corresponding boronic acids are used instead of 9,9-dimethylfluorene-2-boronic acid [8], respectively.

### Example 22

### Method of Producing Exemplified Compound No. 43

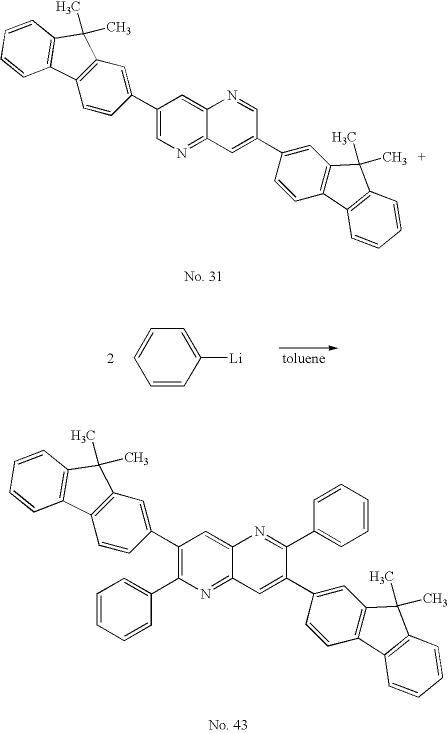

220 mg (0.43 mmol) of Exemplified Compound No. 31 and 20 ml of toluene were loaded into a 100-ml three-necked flask. Then, 2.6 ml (2.6 mmol) of a phenyllithium/cyclohexane-diethyl ether solution [1.04-mol/l] were dropped to the mixture while the mixture was stirred in a nitrogen atmosphere at −78° C. The temperature of the mixture was gradually increased to room temperature, and then the mixture was stirred for 7 hours. After the reaction, the organic layer was extracted with chloroform, dried with anhydrous sodium sulfate, and purified with a silica gel column (mixed developing solvent of chloroform and methanol) and preparative GPC, whereby 71 mg of Exemplified Compound No. 43 (pale yellow crystal) were obtained (25% yield).

### Example 23

### Method of Producing Exemplified Compound No. 47

1 equivalent of 4-methylphenylboronic acid is caused to react with 2,6-dichloro-1,5-naphthyridine [5]. Subsequently, ½ equivalent of 1,4-phenylenediboronic acid is caused to react with the reaction product, whereby Exemplified Compound No. 47 is obtained.

### Example 24

### Method of Producing Exemplified Compound No. 51

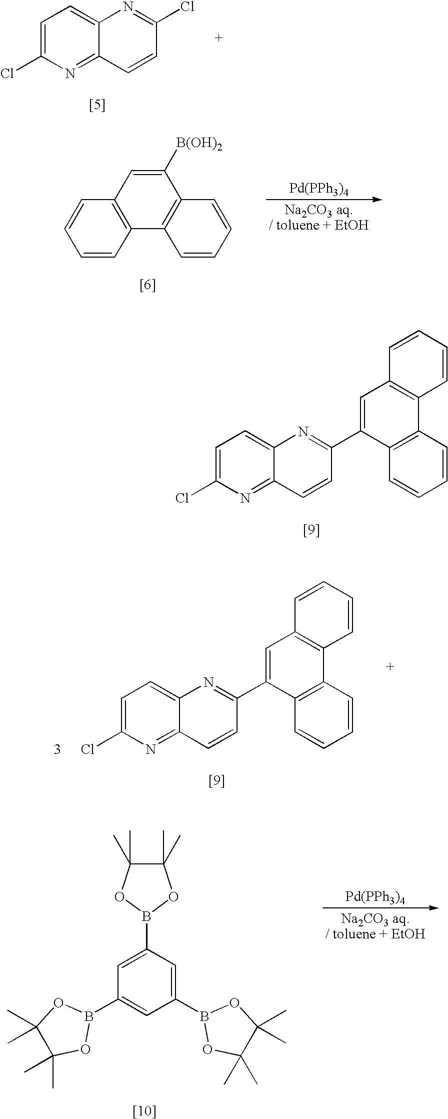

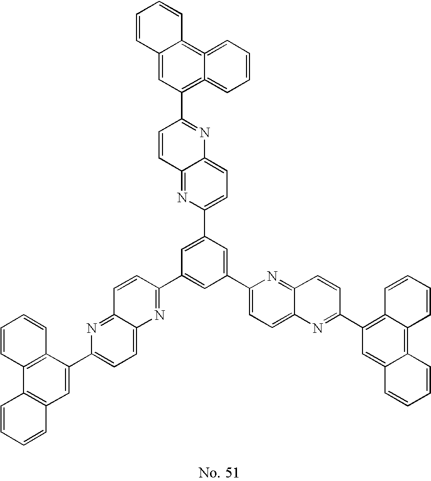

0.22 g of 2-chloro-6-(phenanthren-9-yl)-1,5-naphthyridine [9] was obtained from the following compounds (31% yield) by a Suzuki Coupling reaction similar to the method described in Example 4.

0.50 g (2.5 mmol) of 2,6-dichloro-1,5-naphthyridine [5] 0.47 g (2.1 mmol) of phenanthrene-9-boronic acid [6]

Subsequently, 0.11 g of Exemplified Compound No. 51 (pale yellow crystal) was obtained (68% yield) from 0.22 g (0.65 mmol) of a compound [9] and 73 mg (0.16 mmol) of a tripinacol body [10] by employing the Suzuki Coupling reaction again.

### Example 25

The device having a structure illustrated in FIG. 3 was produced.

Indium tin oxide (ITO) as the anode 2 was formed into a film having a thickness of 120 nm on a glass substrate as the substrate 1 by a sputtering method, and the obtained substance was used as a transparent conductive supporting substrate. The obtained substrate was subjected to ultrasonic cleaning with acetone and isopropyl alcohol (IPA) in the stated order, and was then subjected to boiling cleaning with IPA, followed by drying. Further, the transparent conductive supporting substrate was subjected to UV/ozone cleaning before use.

A solution of a compound represented by the following structural formula in chloroform was formed into a film having a thickness of 20 nm on the transparent conductive supporting substrate by means of a spin coating method, whereby the hole transport layer 5 was formed.

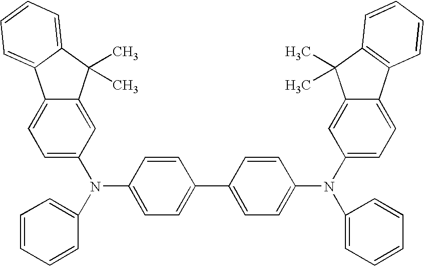

Further, an Ir complex and CBP (at a weight ratio of 5:100) represented by the following structural formulae were formed into a film having a thickness of 20 nm by means of a vacuum deposition method, whereby the light-emitting layer 3 was formed. Film formation was performed under conditions that a degree of vacuum upon deposition was 1.0×10⁻⁴ Pa, and a film forming rate was 0.2 to 0.3 nm/sec.

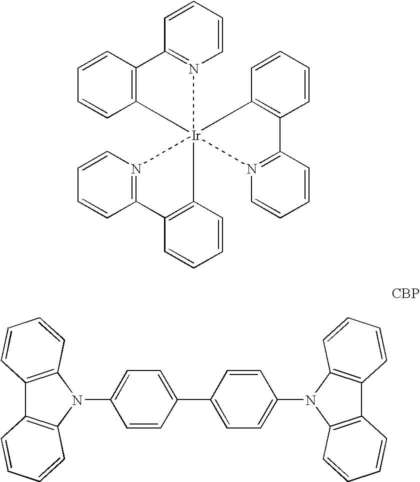

Further, a synthesis of Exemplified Compound No. 4 was formed into a film having a thickness of 40 nm by means of a vacuum deposition method, whereby the electron transport layer 6 was formed. Film formation was performed under conditions that a degree of vacuum upon deposition was 1.0×10⁻⁴ Pa, and a film forming rate was 0.2 to 0.3 nm/sec.

Next, a metal layer film having a thickness of 50 nm as the cathode 4 was formed of a deposition material composed of aluminum and lithium (having a lithium concentration of 1 atomic %) on the organic layer by means of a vacuum deposition method. Further, an aluminum layer having a thickness of 120 nm was formed by means of a vacuum deposition method. Film formation was performed under conditions that a degree of vacuum upon deposition was 1.0×10⁻⁴ Pa, and a film forming rate was 1.0 to 1.2 nm/sec.

Further, the resultant was covered with a protective glass plate and sealed with an acrylic resin-based adhesive in a nitrogen atmosphere.

A direct voltage of 8 V was applied to the device thus obtained in such a manner that the ITO electrode (anode 2) would serve as a positive electrode and the Al—Li electrode (cathode 4) would serve as a negative electrode. As a result, a current flowed in the device at a current density of 15 mA/cm², and green light emission having a luminance of 4,300 cd/m² was observed.

Further, a voltage was applied for 100 hours with a current density kept at 8.0 mA/cm². As a result, a luminance after 100 hours was 900 cd/m² while an initial luminance was 1100 cd/m². This means that luminance degradation was small.

### Examples 26 and 27

In each example, the device was produced in the same manner as in Example 25 except that Exemplified Compound Nos. 1 or 15 were used instead of Exemplified Compound No. 4, and the devices were each evaluated in the same manner as in Example 25. Table 1 shows the results. Further, a voltage was applied for 100 hours with a current density kept at 8.0 mA/cm². As a result, luminance degradation was small.

### Comparative Example 1

The device was produced in the same manner as in Example 25 except that Comparative Compound No. 1 represented by the following structural formula was used instead of Exemplified Compound No. 4, and the device was evaluated in the same manner as in Example 25. Table 1 shows the results. Further, a voltage was applied for 100 hours with a current density kept at 8.0 mA/cm². As a result, a luminance after 100 hours was 420 cd/m² while an initial luminance was 830 cd/m². This result means that luminance degradation was observed.

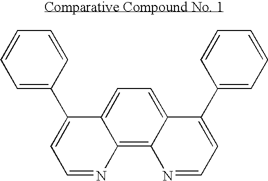

TABLE 1

| | | Initial applied | |
|---|---|---|---|
| | Exemplified | voltage | Luminance |
| Example No. | Compound No. | (V) | (cd/m²) |
| Example 25 | 4 | 8 | 4,300 |
| Example 26 | 1 | 8 | 4,200 |
| Example 27 | 15 | 8 | 4,600 |
| Comparative | Comparative | 8 | 2,800 |
| Example 1 | Compound No. 1 | | |

### Example 28

In the same manner as in Example 25, the hole transport layer 5 was formed on the transparent conductive supporting substrate.

Further, a compound represented by the following structural formula was formed into a film having a thickness of 20 nm by means of a vacuum deposition method, whereby the light-emitting layer 3 was formed. Film formation was performed under conditions that a degree of vacuum upon deposition was 1.0×10⁻⁴ Pa, and a film forming rate was 0.2 to 0.3 nm/sec.

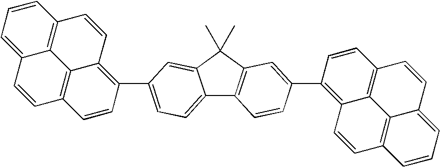

Further, a synthesis of Exemplified Compound No. 2 was formed into a film having a thickness of 20 nm by means of a vacuum deposition method, whereby the electron transport layer 6 was formed. Film formation was performed under conditions that a degree of vacuum upon deposition was 1.0×10⁻⁴ Pa, and a film forming rate was 0.2 to 0.3 nm/sec.

Next, the cathode 4 was formed in the same manner as in Example 25, followed by sealing.

A direct voltage of 7 V was applied to the device thus obtained in such a manner that the ITO electrode (anode 2) would serve as a positive electrode and the Al—Li electrode (cathode 4) would serve as a negative electrode. As a result, a current flowed in the device at a current density of 17 mA/cm², and blue light emission having a luminance of 4,500 cd/m² was observed.

Further, a voltage was applied for 100 hours with a current density kept at 8 mA/cm². As a result, a luminance after 100 hours was 1,500 cd/m² while an initial luminance was 1,900 cd/m². This means that luminance degradation was small.

### Examples 29 and 30

In present examples, the device were produced in the same manner as in Example 28 except that Exemplified Compound Nos. 4 and 15 were used instead of Exemplified Compound No. 2, respectively, and the devices were each evaluated in the same manner as in Example 28. Table 2 shows the results. Further, a voltage was applied for 100 hours with a current density kept at 20 mA/cm². As a result, luminance degradation was small.

### Comparative Example 2

The device was produced in the same manner as in Example 28 except that Comparative Compound No. 1 was used instead of Exemplified Compound No. 2, and the device was evaluated in the same manner as in Example 28. Table 2 shows the results. Further, a voltage was applied for 100 hours with a current density kept at 20 mA/cm². As a result, a luminance after 100 hours was 430 cd/m² while an initial luminance was 730 cd/m². This result means that luminance degradation was observed.

TABLE 2

| | Exemplified | Initial applied | Luminance |
|---|---|---|---|
| Example No. | Compound No. | voltage (V) | (cd/m²) |
| Example 28 | 2 | 7 | 4,500 |
| Example 29 | 4 | 7 | 4,400 |
| Example 30 | 15 | 7 | 4,700 |
| Comparative | Comparative | 7 | 2,400 |
| Example 2 | Compound No. 1 | | |

### Example 31

The device having the structure illustrated in FIG. 2 was produced.

In the same manner as in Example 25, the hole transport layer 5 was formed on the transparent conductive supporting substrate.

Further, Exemplified Compound No. 4 was formed into a film having a thickness of 40 nm by means of a vacuum deposition method, whereby the light-emitting layer and the electron transport layer 6 were formed. Film formation was performed under conditions that a degree of vacuum upon deposition was 1.0×10⁻⁴ Pa; and a film forming rate was 0.2 to 0.3 nm/sec.

Next, the cathode 4 was formed in the same manner as in Example 25, followed by sealing.

A direct voltage of 7 V was applied to the device thus obtained in such a manner that the ITO electrode (anode 2) would serve as a positive electrode and the Al—Li electrode (cathode 4) would serve as a negative electrode. As a result, a current flowed in the device at a current density of 26 mA/cm², and blue light emission having a luminance of 2,400 cd/m² was observed.

Further, a voltage was applied for 100 hours with a current density kept at 15 mA/cm². As a result, a luminance after 100 hours was 1,300 cd/m² while an initial luminance was 1,800 cd/m². This means that luminance degradation was small.

### Examples 32 and 33

In present examples, the device was produced in the same manner as in Example 31 except that Exemplified Compound Nos. 16 and 21 were used instead of Exemplified Compound No. 4, respectively, and the devices were each evaluated in the same manner as in Example 31. Table 3 shows the results. Further, a voltage was applied for 100 hours with a current density kept at 15 mA/cm². As a result, luminance degradation was small.

### Comparative Example 3

The device was produced in the same manner as in Example 31 except that Comparative Compound No. 1 was used instead of Exemplified Compound No. 4, and the device was evaluated in the same manner as in Example 31. Table 3 shows the results. Further, a voltage was applied for 100 hours with a current density kept at 15 mA/cm². As a result, a luminance after 100 hours was 80 cd/m² while an initial luminance was 200 cd/m². This result means that luminance degradation was observed.

TABLE 3

| | Exemplified | Initial applied voltage | Luminance |
|---|---|---|---|
| Example No. | Compound No. | (V) | (cd/m²) |
| Example 31 | 4 | 7 | 2,400 |
| Example 32 | 16 | 7 | 2,500 |
| Example 33 | 21 | 7 | 1,900 |
| Comparative | Comparative | 7 | 420 |
| Example 3 | Compound No. 1 | | |

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2006-312826, filed Nov. 20, 2006, which is hereby incorporated by reference herein in its entirety.

What is claimed is:

1. A 1,5-naphthyridine compound represented by the following general formula [I]: 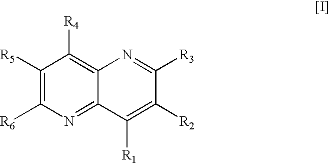
wherein R₁, R₂, R₃, R₄, R₅ and R₆ each represent a group selected from the group consisting of a hydrogen atom, a substituted or unsubstituted alkyl group, a substituted or unsubstituted aralkyl group, a substituted or unsubstituted aryl group, a substituted or unsubstituted heterocyclic group, a substituted or unsubstituted condensed polycyclic aromatic group, a substituted or unsubstituted condensed polycyclic heterocyclic group, a substituted or unsubstituted aryloxy group, a substituted amino group, a halogen atom, a trifluoromethyl group, and a cyano group; and R₁, R₂, R₃, R₄, R₅ and R₆ may be the same as or different from one another, provided that at least two of R₁, R₂, R₃, R₄, R₅ and R₆ each represent a group selected from the group consisting of a substituted or unsubstituted aralkyl group, a substituted or unsubstituted aryl group, a substituted or unsubstituted heterocyclic group, a substituted or unsubstituted condensed polycyclic aromatic group, a substituted or unsubstituted condensed polycyclic heterocyclic group, a substituted or unsubstituted aryloxy group, and a substituted amino group.

2. A 1,5-naphthyridine compound according to claim 1, wherein R₁, R₂, R₄ and R₅ each represent a group selected from the group consisting of a hydrogen atom, a substituted or unsubstituted alkyl group, a halogen atom, a trifluoromethyl group, and a cyano group; and R₃ and R₆ each represent a group selected from the group consisting of a substituted or unsubstituted aralkyl group, a substituted or unsubstituted aryl group, a substituted or unsubstituted heterocyclic group, a substituted or unsubstituted condensed polycyclic aromatic group, a substituted or unsubstituted condensed polycyclic heterocyclic group, a substituted or unsubstituted aryloxy group, and a substituted amino group.

3. A 1,5-naphthyridine compound represented by the following general formula [II]: 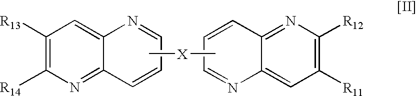
wherein R₁₁, R₁₂, R₁₃ and R₁₄ each represent a group selected from the group consisting of a hydrogen atom, a substituted or unsubstituted alkyl group, a substituted or unsubstituted aralkyl group, a substituted or unsubstituted aryl group, a substituted or unsubstituted heterocyclic group, a substituted or unsubstituted condensed polycyclic aromatic group, a substituted or unsubstituted condensed polycyclic heterocyclic group, a substituted or unsubstituted aryloxy group, a substituted amino group, a halogen atom, a trifluoromethyl group, and a cyano group; and R₁₁, R₁₂, R₁₃ and R₁₄ may be the same as or different from one another; and X represents a group selected from the group consisting of a divalent, substituted or unsubstituted arylene group, a divalent, substituted or unsubstituted heterocyclic group, a divalent, substituted or unsubstituted condensed polycyclic aromatic group, and a divalent, substituted or unsubstituted condensed polycyclic heterocyclic group.

4. A 1,5-naphthyridine compound represented by the following general formula [III]: 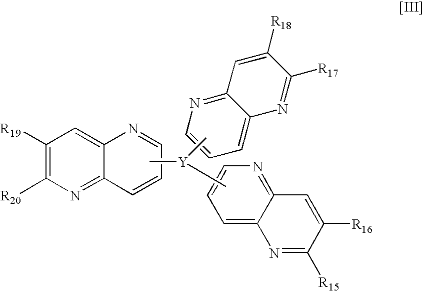
wherein R₁₅, R₁₆, R₁₇, R₁₈, R₁₉ and R₂₀ each represent a group selected from the group consisting of a hydrogen atom, a substituted or unsubstituted alkyl group, a substituted or unsubstituted aralkyl group, a substituted or unsubstituted aryl group, a substituted or unsubstituted heterocyclic group, a substituted or unsubstituted condensed polycyclic aromatic group, a substituted or unsubstituted condensed polycyclic heterocyclic group, a substituted or unsubstituted aryloxy group, a substituted amino group, a halogen atom, a trifluoromethyl group, and a cyano group; and R₁₅, R₁₆, R₁₇, R₁₈, R₁₉ and R₂₀ may be the same as or different from one another; and Y represents a trivalent, substituted or unsubstituted arylene group.

5. An organic light-emitting device, comprising:
a pair of electrodes formed of an anode and a cathode; and
an organic compound layer interposed between the pair of electrodes,
wherein the organic compound layer contains a 1,5-naphthyridine compound according to claim 1.

6. An organic light-emitting device, comprising:
a pair of electrodes formed of an anode and a cathode; and
an organic compound layer interposed between the pair of electrodes,
wherein the organic compound layer contains a 1,5-naphthyridine compound according to claim 3.

7. An organic light-emitting device, comprising:
a pair of electrodes formed of an anode and a cathode; and
an organic compound layer interposed between the pair of electrodes,
wherein the organic compound layer contains a 1,5-naphthyridine compound according to claim 4.

8. An organic light-emitting device according to claim 5, wherein the organic compound layer is one of a light-emitting layer and an electron transport layer.

9. An organic light-emitting device according to claim 6, wherein the organic compound layer is one of a light-emitting layer and an electron transport layer.

10. An organic light-emitting device according to claim 7, wherein the organic compound layer is one of a light-emitting layer and an electron transport layer.
